(19) United States
(12) Patent Application Publication
(10) Pub. No.: US 2009/0075311 A1
(43) Pub. Date: Mar. 19, 2009

(54) ASSESSING COLORECTAL CANCER BY MEASURING HEMOGLOBIN AND M2-PK IN A STOOL SAMPLE

(76) Inventor: Johann Karl, Peissenberg (DE)

Correspondence Address: ROCHE DIAGNOSTICS OPERATIONS INC., 9115 Hague Road, Indianapolis, IN, 46250-0457, US

(21) Appl. No.: 12/140,589

(22) Filed: Jun. 17, 2008

Related U.S. Application Data
(63) Continuation of application No. PCT/EP2006/012217, filed on Dec. 19, 2006.

(30) Foreign Application Priority Data
Dec. 21, 2005 (EP) 05028003.1

(51) Int. Cl. C12Q 1/48; C12Q 1/34; C12Q 1/26; G01N 33/72

(52) U.S. Cl. 435/15; 435/18; 435/25; 436/66

(57) ABSTRACT

The present invention relates to a method aiding in the assessment of colorectal cancer. The method especially is used in assessing the absence or presence of colorectal cancer in vitro. The method is, for example, practiced by analyzing biochemical markers, comprising measuring in a stool sample the concentration of hemoglobin and M2-PK and correlating the concentrations determined to the absence or presence of colorectal cancer. To further improve the assessment of colorectal cancer in a method of this invention the level of one or more additional marker may be determined together with hemoglobin and M2-PK in a stool sample and be correlated to the absence or presence of colorectal cancer. The invention also relates to the use of a marker panel comprising hemoglobin and M2-PK in the early diagnosis of colorectal cancer, and it teaches a kit for performing the method of the invention.

### RELATED APPLICATIONS

This application is a continuation of PCT/EP2006/012217 filed Dec. 19, 2006 and claims priority to EP 05028003.1 filed Dec. 21, 2005.

### FIELD OF THE INVENTION

The present invention relates to a method aiding in the assessment of colorectal cancer. The method especially is used in assessing the absence or presence of colorectal cancer in vitro. The method is, for example, practiced by analyzing biochemical markers, comprising measuring in a stool sample the concentration of hemoglobin and M2-PK and correlating the concentrations determined to the absence or presence of colorectal cancer. To further improve the assessment of colorectal cancer in a method of this invention the level of one or more additional marker may be determined together with hemoglobin and M2-PK in a stool sample and be correlated to the absence or presence of colorectal cancer. The invention also relates to the use of a marker panel comprising hemoglobin and M2-PK in the early diagnosis of colorectal cancer and it teaches a kit for performing the method of the invention.

### BACKGROUND

Stool or fecal samples are routinely tested for the presence of parasites, fat, occult blood, viruses, bacteria and other organisms and chemicals in the diagnosis for various diseases.

Cancer remains a major public health challenge despite progress in detection and therapy. Amongst the various types of cancer, colorectal cancer (=CRC) is one of the most frequent cancers in the Western world.

The staging of cancer is the classification of the disease in terms of extent, progression, and severity. It groups cancer patients so that generalizations can be made about prognosis and the choice of therapy.

Today, the TNM system is the most widely used classification of the anatomical extent of cancer. It represents an internationally accepted, uniform staging system. There are three basic variables: T (the extent of the primary tumor), N (the status of regional lymph nodes) and M (the presence or absence of distant metastases). The TNM criteria are published by the UICC (International Union Against Cancer), Sobin, L. H., Wittekind, Ch. (eds), TNM Classification of Malignant Tumours, fifth edition, 1997.

What is especially important is that early diagnosis of CRC translates to a much better prognosis. Malignant tumors of the colorectum arise from benign tumors, i.e. from adenoma. Therefore, best prognosis have those patients diagnosed at the adenoma stage. Patients diagnosed as early as in stage Tᵢₛ, N0, M0 or T1-3; N0; M0, if treated properly have a more than 90% chance of survival 5 years after diagnosis as compared to a 5-years survival rate of only 10% for patients diagnosed when distant metastases are already present.

In the sense of the present invention the method of assessing the presence or absence of CRC is especially appropriate for the sensitive detection of CRC at a pre-malignant state (adenoma) or at a tumor stage where no metastases at all (neither proximal nor distal), i.e. in UICC classes I, II, or III.

The diagnostic method according to the present invention is based on a stool sample which is derived from an individual. The stool sample is extracted and hemoglobin and M2-PK, respectively is specifically measured from this processed stool sample by use of a specific binding agent.

The earlier cancer can be detected/diagnosed; the better is the overall survival rate. This is especially true for CRC. The prognosis in advanced stages of tumor is poor. More than one third of the patients will die from progressive disease within five years after diagnosis, corresponding to a survival rate of about 40% for five years. Current treatment is only curing a fraction of the patients and clearly has the best effect on those patients diagnosed in an early stage of disease.

With regard to CRC as a public health problem, it is essential that more effective screening and preventative measures for colorectal cancer be developed.

The earliest detection procedures available at present for colorectal cancer involve using tests for fecal blood or endoscopic procedures. However, significant tumor size must typically exist before fecal blood is detected. With regard to detection of CRC from a stool sample, the state of the art has been for quite a while the guaiac-based fecal occult blood test.

The guaiac test is currently most widely used as a screening assay for CRC from stool. The guaiac test, however, has both poor sensitivity as well as poor specificity. The sensitivity of the guaiac-based fecal occult blood tests is ˜26%, which means 74% of patients with malignant lesions will remain undetected (Ahlquist, D. A., Gastroenterol. Clin. North Am. 26 (1997) 41-55). The visualization of precancerous and cancerous lesions represents the best approach to early detection, but colonoscopy is invasive with significant costs, risks, and complications (Silvis, S. E., et al., JAMA 235 (1976) 928-930; Geenen, J. E., et al., Am. J. Dig. Dis. 20 (1975) 231-235; Anderson, W. F., et al., J. Natl. Cancer Institute 94 (2002) 1126-1133).

Stool collection is non-invasive and thus theoretically ideal for testing pediatric or geriatric patients, for testing away from a clinical site, for frequently repeated tests and for determining the presence of analytes which are likely to be found in the digestive tract.

However, the application of immunoassay techniques to analysis of fecal samples has proven to be difficult for several reasons.

Analytes are not distributed throughout the stool specimen but tend to be more concentrated at the outer surface of stool specimen that previously has been in contact with intestinal or even cancerous cells. This is why EP 0 817 968 proposes the use of cross-sectional stool sample for further analysis. The focus of EP 0 817 968 lies in the diagnosis of DNA as comprised in a stool specimen.

Stool handling is disagreeable and biohazardous. Procedures for processing stool have proven to be awkward and frequently complex requiring several handling steps, e.g., filtration or centrifugation. Weighing, extracting, centrifuging, and storing samples are difficult except in a clinical laboratory equipped with suitable apparatuses and skilled technicians.

Analytes in stool samples are frequently unstable; this is believed to be especially true for polypeptides or proteins. Constituents of stool are known to interfere with solid-phase immunoassays. Immunoreactants immobilized on solid-phase may be desorbed by stool constituents. Non-specific reactions may occur.

To increase the commercial use of immunoassay techniques for measuring a proteinaceous analyte in a stool sample, a number of problems must be solved. E.g. analytes have to be solubilized as efficient as possible, the instability of the analyte in the stool has to be dealt with, the interference from stool constituents should be reduced as much as possible, the needs for extensive handling of the stool, equipment contamination, and instrumentation needs must be minimized. Simple preparation steps avoiding the use of expensive equipment and instruments are required to extend the use of immunoassay testing procedures, or at least the sampling procedure for such immunoassay to sites outside hospital and clinical laboratory environments. Examples of stool sample diluents which are of advantage in the detection of proteins like hemoglobin and M2-PK are given further below.

WO 02/18931 discloses a method for preparing stool specimens for diagnostic assays. An improved extraction procedure based on an extraction buffer that essentially comprises a buffer substance, a detergent, preferably a zwitterionic detergent, and a blocking agent is described.

The handling of a stool specimen is facilitated by use of recently developed sampling devices. Appropriate stool sampling devices are e.g. described in EP 1 366 715 and in EP 1 214 447.

Despite the fact that immunological assays for proteins comprised in a stool specimen have been described since the early 1990ies, such assays still are not broadly used in clinical routine. U.S. Pat. No. 5,198,365, for example, describes that it is possible to detect the presence of blood in a stool sample via the specific immunological measurement of hemoglobin.

The sensitivity and specificity of diagnostic alternatives to the guaiac test have been recently investigated by Sieg, A., et al., Int. J. Colorectal Dis. 14 (1999) 267-271. Especially the measurement of hemoglobin and of the hemoglobin-haptoglobin complex from stool specimen have been compared. It has been noted that the hemoglobin assay has an unsatisfactory sensitivity for the detection of a colorectal neoplasm. Whereas cancer in its progressed carcinoma stage is detected with a sensitivity of about 87% the earlier tumor stages are not detected with a sufficient sensitivity. The hemoglobin-haptoglobin complex assay was more sensitive in the detection of earlier stages of CRC. This more sensitive detection was accompanied by a poor specificity. Since poor specificity, however, translates to a high number of unnecessary secondary investigations, like colonoscopy, an assay with a poor accuracy also does not meet the requirements of a generally accepted screening assay.

Recently, an assay for detection of pyruvate kinase M2 isoenzyme (M2-PK) has been introduced into the market (Schebo Biotech, Gieβen, Germany). A comparison of the guaiac assay to the immunoassays for hemoglobin and M2-PK has for example been performed by Vogel, T. et. al., Dtsch. Med. Wochenschr. 130 (2005) 872-877. They show that the immunological assays are superior to the guaiac test and that at comparable specificity the M2-PK assay is less sensitive in detecting CRC as compared to the hemoglobin assay. The authors conclude that the usefulness of both these stool based assays is still questionable.

A further alternative method to the guaiac test for detection of CRC in stool has been published recently and consists in the detection of the colorectal cancer-specific antigen, “minichromosome maintenance protein 2” (MCM2) by immunohistochemistry in colonic cells shed into stool. Due to the small study size, conclusion on the diagnostic value for detection of colorectal cancer is preliminary. However, the test seems to have only limited sensitivity to detect right-sided colon cancer (Davies, R. J., et al., Lancet 359 (2002) 1917-1919).

A need clearly exists to improve the assessment of colorectal cancer.

It was the task of the present invention to find out whether the assessment of CRC, e.g. by use of immunological methods for detection of analytes in a stool specimen can be improved.

It has been found and established that a method for assessing the absence or presence of colorectal cancer in vitro by biochemical markers, comprising measuring in a stool sample the concentration of at least hemoglobin and pyruvate kinase isoform M2 (M2-PK), can help to overcome at least some of the disadvantages mentioned above.

### SUMMARY OF THE INVENTION

The present invention relates to a method for assessing the absence or presence of colorectal cancer in vitro by biochemical markers, comprising measuring in a stool sample the concentration of at least hemoglobin and pyruvate kinase isoform M2 (M2-PK), and correlating the concentrations determined for hemoglobin and M2-PK to the absence or presence of colorectal cancer.

Further the use of a marker panel comprising at least the markers hemoglobin and M2-PK in the diagnosis of colorectal cancer is disclosed.

Also disclosed is a kit for performing the method according to the present invention comprising the reagents required to specifically measure hemoglobin and M2-PK, respectively, and optionally auxiliary reagents for performing the respective measurement.

### BRIEF DESCRIPTION OF THE FIGURE

FIG. 1: ROC-analysis of Hb, M2-PK and a combination of both assays. ROC-analysis of patients diagnosed with CRC (stages I-III, UICC) versus controls (GI-healthy, hemorrhoids, other bowel diseases) and patients with diverticulosis using Hb (continuous line) or M2-PK (short bares) alone or in combination (long bares).

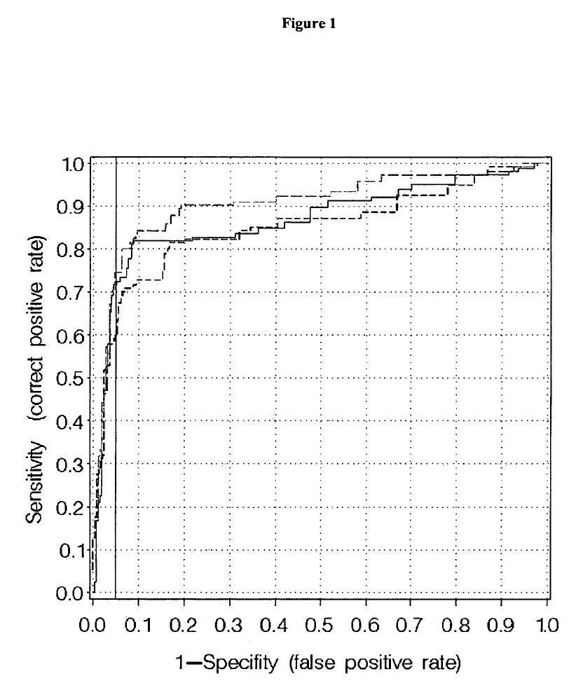

### DETAILED DESCRIPTION OF THE INVENTION

In a first embodiment the present invention relates to a method for assessing the absence or presence of colorectal cancer in vitro by biochemical markers, comprising measuring in a stool sample the concentration of at least (a) hemoglobin and (b) pyruvate kinase isoform M2 (M2-PK), and (c) correlating the concentrations determined in steps (a) and (b) to the absence or presence of colorectal cancer.

The term “assessing colorectal cancer” is used to indicate that the method according to the present invention will, together with other variables, e.g., the confirmation by colonoscopy, aid the physician to establish a diagnosis of colorectal cancer (CRC). In a preferred embodiment, this assessment will relate to the presence or absence of CRC. As the skilled artisan will appreciate, no single biochemical marker and no marker combination is diagnostic with 100% specificity and at the same time 100% sensitivity for a given disease, rather biochemical markers are used to assess with a certain likelihood or predictive value the presence or absence of a disease. Preferably the method according to the present invention aids in assessing the presence or absence of CRC.

As the skilled artisan will appreciate, the step of correlating a marker level to the presence or absence of CRC can be performed and achieved in different ways. In general a reference population is selected and a normal range established. It is no more than routine experimentation, to establish the normal range for both hemoglobin as well as M2-PK-levels in stool samples by using an appropriate reference population. It is generally accepted that the normal range to a certain but limited extent depends on the reference population in which it is established. The ideal and preferred reference population is high in number, e.g., hundreds to thousands and matched for age, gender and optionally other variables of interest. The normal range in terms of absolute values, like a concentration given, also depends on the assay employed and the standardization used in producing the assay.

The levels given for hemoglobin and M2-PK in the examples section have been measured from aliquots derived from the same stool sample and established with the assay procedures given.

In a method according to the present invention at least the concentration of the biomarkers hemoglobin and M2-PK, respectively, as present in a stool sample is determined and the marker combination is correlated to the absence or presence of CRC.

As the skilled artisan will appreciate there are many ways to use the measurements of two or more markers in order to improve the diagnostic question under investigation. In a quite simple, but nonetheless often effective approach, a positive result is assumed if a sample is positive for at least one of the markers investigated. This may e.g. be the case when diagnosing an infectious disease, like ADS. Frequently, however, the combination of markers is evaluated. Preferably the values measured for markers of a marker panel, e.g. for hemoglobin and M2-PK, are mathematically combined and the combined value is correlated to the underlying diagnostic question.

Marker values may be combined by any appropriate state of the art mathematical method. Well-known mathematical methods for correlating a marker combination to a disease employ methods like, Discriminant analysis (DA) (i.e. linear-, quadratic-, regularized-DA), Kernel Methods (i.e. SVM), Nonparametric Methods (i.e. k-Nearest-Neighbor Classifiers), PLS (Partial Least Squares), Tree-Based Methods (i.e. Logic Regression, CART, Random Forest Methods, Boosting/Bagging Methods), Generalized Linear Models (i.e. Logistic Regression), Principal Components based Methods (i.e. SIMCA), Generalized Additive Models, Fuzzy Logic based Methods, Neural Networks and Genetic Algorithms based Methods. The skilled artisan will have no problem in selecting an appropriate method to evaluate a marker combination of the present invention. Preferably the method used in correlating the marker combination of the invention e.g. to the absence or presence of CRC is selected from DA (i.e. Linear-, Quadratic-, Regularized Discriminant Analysis), Kernel Methods (i.e. SVM), Nonparametric Methods (i.e. k-Nearest-Neighbor Classifiers), PLS (Partial Least Squares), Tree-Based Methods (i.e. Logic Regression, CART, Random Forest Methods, Boosting Methods), or Generalized Linear Models (i.e. Logistic Regression). Details relating to these statistical methods are found in the following references: Ruczinski, I., et al., J. of Computational and Graphical Statistics 12 (2003) 475-511; Friedman, J. H., J. of the American Statistical Association 84 (1989) 165-175; Hastie, T., et al., The Elements of Statistical Learning, Springer Verlag (2001); Breiman, L., et al., Classification and regression trees, California, Wadsworth (1984); Breiman, L., Machine Learning 45 (2001) 5-32; Pepe, M. S., The Statistical Evaluation of Medical Tests for Classification and Prediction, Oxford Statistical Science Series, 28 (2003); and Duda, R. O., et al., Pattern Classification, Wiley Interscience, 2nd edition (2001).

It is a preferred embodiment of the invention to use an optimized multivariate cut-off for the underlying combination of biological markers and to discriminate state A from state B, e.g. presence of CRC from absence of CRC. In this type of analysis the markers are no longer independent but form a marker panel. It could be established that combining the measurements of hemoglobin and of M2-PK does significantly improve the diagnostic accuracy for CRC as compared to healthy controls. This becomes especially evident if only samples obtained from patients with early stages of CRC (UICC stages I to III) are included in the analysis. Especially the later finding is of great importance, because patients with early CRC are likely to profit most from a correct and early detection of a malignancy.

Accuracy of a diagnostic method is best described by its receiver-operating characteristics (ROC) (see especially Zweig, M. H., and Campbell, G., Clin. Chem. 39 (1993) 561-577). The ROC graph is a plot of all of the sensitivity/specificity pairs resulting from continuously varying the decision thresh-hold over the entire range of data observed.

The clinical performance of a laboratory test depends on its diagnostic accuracy, or the ability to correctly classify subjects into clinically relevant subgroups. Diagnostic accuracy measures the test's ability to correctly distinguish two different conditions of the subjects investigated. Such conditions are for example health and disease or benign versus malignant disease.

In each case, the ROC plot depicts the overlap between the two distributions by plotting the sensitivity versus 1−specificity for the complete range of decision thresholds. On the y-axis is sensitivity, or the true-positive fraction [defined as (number of true-positive test results)/(number of true-positive+number of false-negative test results)]. This has also been referred to as positivity in the presence of a disease or condition. It is calculated solely from the affected subgroup. On the x-axis is the false-positive fraction, or 1−specificity [defined as (number of false-positive results)/(number of true-negative+number of false-positive results)]. It is an index of specificity and is calculated entirely from the unaffected subgroup.

Because the true- and false-positive fractions are calculated entirely separately, by using the test results from two different subgroups, the ROC plot is independent of the prevalence of disease in the sample. Each point on the ROC plot represents a sensitivity/1−specificity pair corresponding to a particular decision threshold. A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination (identical distributions of results for the two groups) is a 45° diagonal line from the lower left corner to the upper right corner. Most plots fall in between these two extremes. (If the ROC plot falls completely below the 45° diagonal, this is easily remedied by reversing the criterion for “positivity” from “greater than” to “less than” or vice versa.) Qualitatively, the closer the plot is to the upper left corner, the higher the overall accuracy of the test.

One convenient goal to quantify the diagnostic accuracy of a laboratory test is to express its performance by a single number. The most common global measure is the area under the ROC plot (area under the curve=AUC). By convention, this area is always ≧0.5 (if it is not, one can reverse the decision rule to make it so). Values range between 1.0 (perfect separation of the test values of the two groups) and 0.5 (no apparent distributional difference between the two groups of test values). The area does not depend only on a particular portion of the plot such as the point closest to the diagonal or the sensitivity at 90% specificity, but on the entire plot. This is a quantitative, descriptive expression of how close the ROC plot is to the perfect one (area=1.0).

In a preferred embodiment the present invention relates to a method for improving the diagnostic accuracy for colorectal cancer versus controls by measuring in a sample the concentration of at least hemoglobin and M2-PK and correlating the concentrations determined to the presence or absence of CRC, the improvement resulting in more patients being correctly classified as suffering from CRC versus healthy controls as compared to a classification based on either marker alone. The CRC marker panel comprising hemoglobin and M2-PK can of course also be used in assessing the severity of disease for patients suffering from CRC.

As the skilled artisan will appreciate one or more additional biomarker may be used to further improve the assessment of CRC. To illustrate this additional potential of using hemoglobin and M2-PK as the key markers of a panel of markers for assessment of CRC the term “at least” has been used in the appending claims. With other words, the level measured for one or more additional marker may be combined with the measurement of hemoglobin and M2-PK in the assessment of CRC.

The one or more additional marker used together with hemoglobin and M2-PK may be considered to be part of a CRC marker panel, i.e., a series of markers appropriate to further refine the assessment of CRC. The total number of markers in a CRC marker panel is preferably less than 20 markers, more preferred less than 15 markers, also preferred are less than 10 markers with 8 or less markers being even more preferred. Preferred are CRC marker panels comprising 3, 4, 5, or 6 markers in total.

In a preferred embodiment the present invention thus relates to a method for assessing the absence or presence of colorectal cancer in vitro by biochemical markers, comprising measuring in a sample the concentration of hemoglobin and M2-PK and in addition the concentration of one or more other marker and correlating the concentrations of hemoglobin, M2-PK, and of the one or more additional marker to the absence or presence of colorectal cancer.

Hemoglobin, like any abundant serum protein may be considered to be indicative for the extend of bleeding caused by a cancerous lesion. It is therefore envisaged and preferred that another highly abundant serum proteins, i.e. a serum protein present at a concentration of 1 mg/ml or above (e.g. serum albumin) is used as a substitute marker for hemoglobin.

Preferably the one or more other marker is selected from the group consisting of CEA, CYFRA 21-1, CA19-9, CA724, NNMT, PROC, and SAHH.

In a preferred embodiment the present invention the method of assessing the presence or absence of colorectal cancer is based on the measurement of at least hemoglobin, M2-PK, and SAHH.

An assay for “CYFRA 21-1” specifically measures a soluble fragment of cytokeratin 19 as present in the circulation. The measurement of CYFRA 21-1 is typically based upon two monoclonal antibodies (Bodenmueller, H., et al., Int. J. Biol. Markers 9 (1994) 75-81). In the CYFRA 21-1 assay from Roche Diagnostics, Germany, the two specific monoclonal antibodies (KS 19.1 and BM 19.21) are used and a soluble fragment of cytokeratin 19 having a molecular weight of approx. 30,000 Daltons is measured.

The carbohydrate antigen 19-9 (CA 19-9) values measured are defined by the use of the monoclonal antibody 1116-NS-19-9. The 1116-NS-19-9-reactive determinants on a glycolipid having a molecular weight of approx. 10,000 Daltons are measured. This mucin corresponds to a hapten of Lewis-a blood group determinants and is a component of a number of mucous membrane cells. (Koprowski, H., et al., Somatic Cell Genet 5 (1979) 957-972). CA 19-9 can e.g., be measured on the ELECSYS analyzer (Roche Diagnostics GmbH) using Roche product number 11776193 according to the manufacturers instructions.

Carcinoembryonic antigen (CEA) is a monomeric glycoprotein (molecular weight approx. 180,000 Dalton) with a variable carbohydrate component of approx. 45-60% (Gold, P. and Freedman. S. O., J. Exp. Med. 121 (1965) 439-462). High CEA concentrations are frequently found in cases of colorectal adenocarcinoma (Fateh-Moghadam, A., and Stieber, P., Sensible use of tumor markers, Boehringer Mannheim, Cat. No. 1536869 (Engl.), 1320947 (German), ISBN 3-926725-07-9 German/English, Juergen Hartmann Verlag GmbH, Marloffstein-Rathsberg (1993). Slight to moderate CEA elevations (rarely >10 ng/mL) occur in 20-50% of benign diseases of the intestine, the pancreas, the liver, and the lungs (e.g. liver cirrhosis, chronic hepatitis, pancreatitis, ulcerative colitis, Crohn's Disease, emphysema) (Fateh-Moghadam, A., and Stieber, P., supra). Smokers also have elevated CEA values. The main indication for CEA determinations is the follow-up and therapy management of colorectal carcinoma.

The protein nicotinamide N-methyltransferase (NNMT; Swiss-PROT: P40261) has an apparent molecular weight of 29.6 kDa and an isoelectric point of 5.56. It has recently been found (WO 2004/057336) that NNMT will be of interest in the assessment of CRC. The immunoassay described in WO 2004/057336 has been used to measure the samples (CRC, healthy controls and non-malignant colon diseases) of the present study.

The protein pyrroline-5-carboxylate reductase (PROC; Swiss-PROT: P32322) is also known as PYCR1 in the literature. PROC catalyzes the NAD(P)H-dependent conversion of pyrroline-5-carboxylate to proline. Merrill, M. J., et al., J. Biol. Chem. 264 (1989) 9352-9358 studied the properties of human erythrocyte pyrroline-5-carboxylate reductase. They concluded that in addition to the traditional role of catalyzing the obligatory and final unidirectional step in pyrroline biosynthesis, the enzyme may play a physiologic role in the generation of NADP(+) in some cell types including human erythrocytes. PROC has recently been identified as a marker of CRC (WO 2005/095978).

A great part of all tumors expresses the pyruvate kinase glycolytic enzyme isoform (M2-PK). M2-pyruvate kinase occurs in both a tetrameric form which shows a high affinity for the substrate phosphoenolpyruvate (PEP), and the dimeric form, which has a low affinity for PEP. The dimeric form predominates in tumors and was therefore named tumor M2-PK by Eigenbrodt, E., et al., Crit. Rev. Oncog. 3 (1992) 91-115. In a large clinical study conducted by Hardt, P. D., et al., Br. J. Cancer 91 (2004) 980-984) at the Giessen University Hospital the usefulness of the Tumor M2-PK stool test has been evaluated. They reported a sensitivity of 73% for the Tumor M2-PK stool test, combined with a specificity of 78%.

The protein SAHH (S-adenosylhomocysteine hydrolase; SWISS-PROT: P23526) has recently been identified as a marker of colorectal cancer (WO2005/015221). The corresponding cloned human cDNA encodes for a 48-kDa protein. SAHH catalyzes the following reversible reaction: S-adenosyl-L-homocysteine+H2O [symbol] adenosine+L-homocysteine (Cantoni, G. L., Annu. Rev. Biochem. 44 (1975) 435-451). Hershfield and Francke (Hershfield, M. S. and Francke, U., Science 216 (1982) 739-742) located the corresponding gene to chromosome 20 and later on Coulter-Karis and Hershfield (Coulter-Karis, D. E. and Hershfield, M. S., Ann. Hum. Genet. 53 (1989) 169-175) sequenced the full-length cDNA. Recently, the structure of SAHH has been resolved (Turner, M. A., et al., Cell. Biochem. Biophys. 33 (2000) 101-125).

As the skilled artisan will appreciate one or more additional, marker may be used to further improve the diagnostic accuracy, or, where required increase the diagnostic sensitivity at the expense of specificity or vice versa. In some diagnostic areas, e.g., in the detection of an HIV-infection sensitivity is of utmost importance. The high sensitivity required may be achieved at the expense of specificity, leading to an increased number of false positive cases. In other cases, e.g. as a simple example, when assessing blood group antigens, specificity is of paramount importance.

The method according to the present invention appears to be suitable for screening asymptomatic individuals for the presence or absence of CRC. In doing so, both specificity as well as sensitivity are of paramount importance. It is generally accepted that a method used in the screening for a disease with low prevalence, like CRC, the specificity has to be at least 90%, preferably even 95%. With other words, in the latter case the false positive fraction would be 5% or less. This means that not too many costly follow-up examinations are inadvertently caused at such level of specificity. Preferably the method for assessing the absence or presence of colorectal cancer in vitro by biochemical markers according to the present invention has a specificity of at least 90%, even more preferred of 95%.

The method for assessing the absence or presence of colorectal cancer in vitro by measuring at least hemoglobin and M2-PK in a stool sample according to the present invention at a fixed level of specificity of 95% has an improved sensitivity for detection of CRC.

A further preferred embodiment relates to the use of a marker panel in the diagnosis of CRC the panel comprising hemoglobin and M2-PK. Further preferred is the use of a marker panel comprising hemoglobin, M2-PK, and at least one additional marker selected from the group consisting of CEA, CYFRA 21-1, CA19-9, CA724, NNMT, PROC, and SAHH.

A preferred marker panel according to the present invention will comprise the markers hemoglobin, M2-PK, and SAHH.

In a preferred embodiment the method according to the present invention for assessing the absence or presence of colorectal cancer in vitro by biochemical markers that comprises measuring in a stool sample the concentration of at least hemoglobin and pyruvate kinase isoform M2 (M2-PK), makes use of a special new diluent for stool samples described in some detail below.

A preferred stool sample diluent will at least comprise a buffer, a protease inhibitor, and a non-ionic detergent. The buffer in certain preferred embodiments additionally comprises a blocking agent and/or a preservative.

The skilled artisan is familiar with appropriate buffer systems. Preferably the buffer or buffer system will be selected from the group consisting of phosphate buffered saline (PBS), Tris-Hydroxymethylaminoethane (Tris) buffered saline (TBS), N-(2-hydroxyethyl)-piperazine-N′-2-ethanesulfonic acid (HEPES), and 3-(N-Morpholino) propanesulfonic acid (MOPS). Preferably the buffer will have a molarity of between 20 and 200 mM.

The pH of the stool sample diluent preferably is adjusted to a pH-value between pH 6.5 and pH 8.5, more preferably to a pH-value between pH 7.0 and pH 8.0, and further preferred to a pH-value between pH 7.2 and pH 7.7. The skilled artisan will have no difficulty in selecting the appropriate concentration of the buffer constituents in order to ensure that after diluting and mixing the stool specimen with the stool sample diluent the desired pH is attained.

The stool sample diluent comprises a protease inhibitor. There is an ever increasing number of proteases and also of corresponding protease inhibitors.

One important class of proteases are the so-called serine proteases that have the amino acid serine in their active site. Well-known examples of serine proteases are trypsin, chymotrypsin, kallikrein, and urokinase. The skilled artisan is familiar with the fact that certain protease inhibitors are active against serine proteases. The inhibitory potential of such proteases and their activity spectrum is e.g. described in the data sheets from commercial suppliers, like Serva, Heidelberg, or Roche Diagnostics GmbH, Mannheim. Preferably the serine protease inhibitor is selected from the group consisting of AEBSF-HCl (e.g., Serva Cat.No. 12745), APMSF-HCl (e.g., Serva Cat.No. 12320), aprotinin (e.g., Roche Diagnostics, Cat.No. 10 981 532 001), chymostatin (e.g., Roche Diagnostics, Cat.No. 11 004 638 001), PEFABLOC SC (Pentapharm Ltd.) (e.g., Roche Diagnostics, Cat.No. 11 585 916 001), and PMSF (e.g., Roche Diagnostics, Cat.No. 10 837 091 001).

A further important class of proteases are the so-called cysteine proteases that have the amino acid cysteine in their active site. Well-known examples of cysteine proteases are papain and calpain. The skilled artisan is familiar with the fact that certain protease inhibitors are active against cysteine proteases. Some of these inhibitors are also active against serine proteases, e.g., PMSF may be used as an inhibitor of cysteine proteases as well as an inhibitor of serine proteases. The inhibitory potential of such proteases and their activity spectrum is e.g. described in the data sheets from commercial suppliers, like Serva, Heidelberg, or Roche Diagnostics GmbH, Mannheim. Preferably the cysteine protease inhibitor is selected from the group consisting of leupeptine (e.g., Roche Diagnostics, Cat.No. 11 034 626 001), PMSF (see above), and E-64 (e.g., Roche Diagnostics, Cat.No. 10 874 523 001).

A further important class of proteases are the so-called metalloproteases. Metalloproteases are characterized by containing a metal ion e.g., Zn²⁺, Ca²⁺ or Mn²⁺ in the active center. Well-known examples of metalloproteases are digestive enzymes such as carboxypeptidases A and B and thermolysin. The skilled artisan is familiar with the fact that certain protease inhibitors are active against metalloproteases. Metalloproteases are most easily inactivated by substances binding to the metal ion and forming a metal chelate complex therewith. Preferably ethylene-diaminotetra acetic acid (EDTA), ethyleneglycol bis(aminoethylether)tetra acetic acid (EGTA), and/or 1,2-diaminocyclohexane-N,N,N′,N′-tetra acetic acid (CDTA) are used to inactivate metalloproteases. Other appropriate inhibitors of metalloproteases are Phosphoramidon (=N-(α-Rhamnopyranosyloxyhydroxyphosphinyl)-L-leucyl-Ltryptophan, disodium salt; e.g., Roche Diagnostics Cat.No. 10 874 531 001) and bestatin (e.g., Roche Diagnostics Cat.No. 10 874 515 001). The inhibitory potential of these protease inhibitors and their activity spectrum is e.g. described in the corresponding data sheets from commercial suppliers, like Serva, Heidelberg, or Roche Diagnostics GmbH, Mannheim. Preferred inhibitors of metalloproteases are EDTA, EGTA and/or bestatin.

A further important class of proteases is known as aspartic (acidic) proteases. Aspartic proteases are characterized by having an aspartic acid residue in the active center. Well-known examples of aspartic proteases are pepsin, cathepsin D, chymosin, and renin. The skilled artisan is familiar with the fact that certain protease inhibitors are active against aspartic proteases. Preferred inhibitors of aspartic acid proteases are α2-macroglobulin (e.g, Roche Diagnostics Cat.No. 10 602 442 001) and pepstatin (e.g, Roche Diagnostics Cat.No. 11 359 053 001).

For certain applications it will be possible to apply the method according to the present invention by using a stool sample diluent that comprises only one protease inhibitor that protects the polypeptide of interest by e.g. blocking a certain class of proteases.

Preferably the stool sample diluent will comprise at least two different protease inhibitors with activity against two classes of proteases selected from the group, consisting of serine proteases, cysteine proteases, metalloproteases and aspartic proteases. Also preferred at least three of these enzyme classes will be inhibited by an appropriate inhibitor cocktail. Preferably the stool sample diluent will contain a protease inhibitor cocktail that is composed of protease inhibitors that are active against serine proteases, cysteine proteases, metalloproteases and aspartic proteases, respectively.

Preferably at most 20 different protease inhibitors will be used to set up a protease inhibitor cocktail for a stool sample diluent. Also preferred no more than 15 different protease inhibitors will be used. Preferably 10 or less different protease inhibitors as contained in a stool diluent, will suffice to achieve sufficient protease inhibition in order to stabilize a proteinaceous analyte in a stool sample.

Preferably the protease inhibitor is selected from the group consisting of aprotinin, chymostatin, leupeptine, EDTA, EGTA, CDTA, pepstatin A, phenylmethylsulfonyl fluoride (PMSF), and PEFABLOC SC. Preferably the protease inhibitor cocktail contains chymostatin, leupeptine, CDTA, pepstatin A, PMSF, and PEFABLOC SC, also preferred a protease inhibitor cocktail containing aprotinin, leupeptine, EDTA and PEFABLOC SC is used.

A preferred stool sample diluent also comprises a nonionic detergent. Detergents are usually classified into anionic detergents, cationic detergents, amphiphilic detergents and nonionic detergents. The detergent optimal for use in a stool sample diluent according to the present invention must be capable of releasing the analyte of interest from the sample and at the same time it should allow for stabilization of the analyte. This tightrope walk surprisingly can be accomplished by use of a nonionic detergent. Preferably the nonionic detergent used in a stool sample diluent according to the present invention is selected from the group consisting of BRIJ 35 (ICI Americas Inc.), TWEEN 20 (ICI Americas Inc.), Thesit, TRITON X100 (Union Carbide), and Nonidet P40. Amongst the nonionic detergents tested, a stool sample diluent containing Nonidet P40 had the tendency to yield quite satisfactory results. Therefore an appropriate stool sample diluent preferably will contain Nonidet P40 as non-ionic detergent.

The skilled artisan will have no difficulty in selecting an appropriate concentration for the nonionic detergent. He will select a concentration that, after mixture with the stool sample is at or above the critical micelle concentration (CMC). Preferably the concentration of the nonionic detergent in the stool sample diluent is the range of 0.01 to 1 wt. % and also preferably from 0.02 to 0.5 wt. %.

The stool sample diluent preferably also comprises a blocking agent. Many blocking agents are known from the relevant art, like animal proteins or enzymatically generated peptide fragments thereof. Preferably the blocking agent according to this invention will be a serum albumin, casein, a skimmed milk powder, or a digest of an animal protein e.g. a peptone. Preferably the blocking agent is selected from the group consisting of bovine serum albumin (BSA), skimmed milk powder, and chicken albumen. The concentration of the blocking agent can be from 0.1 to 10 wt. % and is preferably from 0.1 to 5 wt. %.

A preferred stool sample diluent comprises a buffer, a protease inhibitor, a blocking agent, and a non-ionic detergent. The stool sample diluent additionally may comprise a preservative. Such preservative preferably is selected form the group consisting of sodium azide, oxy-pyrion, and N-methylisothiazolon.

Most procedures using a stool specimen as a sample require the direct transfer of the stool specimen to the test system, e.g., to the test areas of a guaiac test. Transfer of, e.g., hemoglobin from the sample to the test system is only partial. Undesirable reactions caused by stool constituents are difficult to control with reagents due to their uniform distribution throughout the sample. Most of the procedures require a well equipped laboratory and trained technicians.

The less handling steps and the more robust the sampling and extraction of a stool sample the better.

Several recent developments have focused on device that facilitate the sampling and handling of a stool sample. EP 1 366 715 discloses a special collection tube for collection of a stool sample. This extraction tube essentially comprises (a) a container body that is hollow on the inside, open at the top, and able to receive a buffer solution, (b) a top cap provided with a threaded small rod for collection of fecal samples, said threaded small rod protruding axially inside the container body, when the top cap is applied to the top end of the container body, and (c) a dividing partition provided, in an intermediate position, inside said container body so as to separate a top chamber from a bottom chamber inside said container body, said dividing partition having an axial hole suitable to allow the passage of said threaded small rod, so as to retain the excess feces in said top chamber and allow the passage of the threaded part of the small rod into said bottom chamber. This extraction tube further has a container body that is open at the bottom and provided with a bottom cap which can be applied movably to the bottom end of the container body, so that said extraction tube can be used directly as a primary sampling tube to be inserted into a sample-holder plate of automatic analyzers, following removal of said bottom cap and overturning of said container body. With more simple words the tube disclosed in EP 1 366 715 allows for a convenient handling of a defined quantity of a stool sample and has the advantage that after appropriate extraction the tube may be directly placed into the sample-holder of an automatic analyzer. The reader will find the detailed disclosure of this stool sampling tube in the above captioned patent, the full disclosure is herewith incorporated by reference.

In WO 03/068398 a sophisticated stool sampling device is described that also is appropriate for a convenient sampling and handling of a stool sample. The features of the device disclosed in this WO-application are explicitly referred to and herewith enclosed by reference in their entirety. In WO 03/069343 it is recommended to extract a stool specimen, e.g., collected with a device according to WO 03/068398 by use of an extraction buffer comprising 10 mM CHAPS (=3-[(3-chloramidopropyl)-dimethylammonio]-1-propanesulfonate), which is a zwitterionic detergent.

For preparing a fecal sample composition for immunoassay testing a dispersion of at most 10 wt. %, preferably from 0.1 wt. % to up to 10 wt. % and more preferably from 0.5 to 5 wt. % of a stool sample in the stool sample diluent is made. Preferably the mixing of the stool sample with the diluent is made directly within an appropriate sampling tube already prefilled with a stool sample diluent as described above.

The stool sample is preferably freshly collected and given into the stool sample diluent directly. No intermediate storage, transportation and/or handling is necessary.

The level of hemoglobin and M2-PK, respectively, is detected by any appropriate assay method. In clinical routine such methods in most cases will employ antibodies to the target antigen, the so-called immunoassays. A wide variety of immunoassay procedures including latex agglutination, competition and sandwich immunoassays can be carried out for detecting a proteinaceous analyte in a stool sample if such stool sample is e.g., prepared as described in detail above.

The immunoassay used preferably is a heterogeneous immunoassay. It is also preferred that the detection of the proteinaceous analyte is accomplished by aid of a competitive immunoassay or by aid of a so-called sandwich immunoassay.

The skilled artisan will have no problem in setting up an immunoassay which is capable of detecting the target antigen or target analyte as present in the extract of a stool sample.

By way of example such detection may be performed in a sandwich type immunoassay. Typically a first anti-analyte antibody is directly or indirectly bound to a solid phase. With other words, the first antibody binding to the target antigen is used as a capture antibody. For determining a target analyte, e.g. in an extract of a human stool sample the extract is incubated under appropriate conditions and for a time sufficient to permit a binding of the capture antibody to the analyte. For detection of the target antigen a second or detection antibody to the target antigen which binds to an epitope different to the one recognized by the capture antibody is used. Incubation with this second antibody may be performed before, after or at the same time as the incubation with the first antibody.

Preferably the detection antibody is labeled in such a manner that direct or indirect detection is facilitated.

For direct detection the labeling group can be selected from any known detectable marker groups, such as dyes, luminescent labeling groups such as chemiluminescent groups, e.g., acridinium esters or dioxetanes, or fluorescent dyes, e.g., fluorescein, coumarin, rhodamine, oxazine, resorufin, cyanine and derivatives thereof. Other examples of labeling groups are luminescent metal complexes, such as ruthenium or europium complexes, enzymes, e.g., as used for ELISA or for CEDIA (Cloned Enzyme Donor Immunoassay, e.g., EP 0 061 888), and radioisotopes.

Indirect detection systems comprise, for example, that the detection reagent, e.g., the detection antibody is labeled with a first partner of a bioaffine binding pair. Examples of suitable binding pairs are hapten or antigen/antibody, biotin or biotin analogues such as aminobiotin, iminobiotin or desthiobiotin/avidin or streptavidin, sugar/lectin, nucleic acid or nucleic acid analogue/complementary nucleic acid, and receptor/ligand, e.g., steroid hormone receptor/steroid hormone. Preferred first binding pair members comprise hapten, antigen and hormone. Especially preferred are haptens like digoxin and biotin and analogues thereof. The second partner of such binding pair, e.g. an antibody, streptavidin, etc., usually is labeled to allow for direct detection, e.g., by the labels as mentioned above.

Immunoassays are well known to the skilled artisan. Methods for carrying out such assays as well as practical applications and procedures are summarized in related textbooks. Examples of related textbooks are Tijssen, P., Preparation of enzyme-antibody or other enzyme-macromolecule conjugates, In: Practice and theory of enzyme immunoassays, Burdon, R. H. and v. Knippenberg, P. H. (eds.), Elsevier, Amsterdam (1990), pp. 221-278), and various volumes of Methods in Enzymology, Colowick, S. P. and Caplan, N. O. (eds.), Academic Press), dealing with immunological detection methods, especially volumes 70, 73, 74, 84, 92 and 121.

Based on the stool sample diluent described above, it is possible to handle a stool sample in a very convenient manner. Preferably at least one of the markers hemoglobin or M2-PK is detected from a stool sample collected in a stool sample diluent as described above. Preferably both analytes are detected from a stool sample collected in a stool sample diluent as described above. It is also preferred to use the preferred compositions of such a stool sample diluent in the detection of either M2-PK or hemoglobin, or in the detection of both these analytes.

The present invention also relates to a kit for performing the method of this invention comprising the reagents required to specifically measure hemoglobin and M2-PK, respectively.

In yet a further preferred embodiment the kit will comprise reagents required for performing the measurement of both hemoglobin and M2-PK and in addition a stool sampling device, prefilled with an appropriate stool sample diluent.

The following examples and figure are provided to aid the understanding of the present invention, the true scope of which is set forth in the appended claims. It is understood that modifications can be made in the procedures set forth without departing from the spirit of the invention.

### EXAMPLE 1

### Study Population

Stool samples derived from 94 well-characterized CRC patients with the UICC classification given in table 1 have been used.

TABLE 1
CRC samples and corresponding UICC classification

| Stage according to UICC | Number of samples |
|---|---|
| UICC 0 | 2 |
| UICC I | 21 |
| UICC II | 27 |
| UICC III | 36 |
| UICC I-III (non-IV, classes I to III not | 8 |
| separately staged) | |
| Total number of CRC | 94 |

The CRC samples of table 1 have been evaluated in comparison to control samples obtained from individuals, who underwent a colonoscopy and had no adenomas, polyps or colorectal cancers. Table 2 gives an overview of the controls used:

TABLE 2
Composition of the control group

| Type of control patients | Number of samples |
|---|---|
| Healthy controls (no evidence of any | 32 |
| bowel disease) | |
| Hemorrhoids | 89 |
| Diverticulosis | 117 |
| Other bowel diseases | 15 |
| Total number of controls | 253 |

### EXAMPLE 2

### Extraction of the Stool Samples for the Determination of Hemoglobin and M2-PK

For each patient investigated, two stool aliquots were collected at different sites of a single feces before a colonoscopy or a surgery was performed. Approx. 1 g in total per stool sample was collected using a specific collecting device from Sarstedt (Id. no. 80.623.022). The such collected stool specimen were frozen as soon as possible and stored at −70° C. until extraction.

For the determination of hemoglobin 100 mg of the stool specimen was given into a 2 ml Eppendorf-cup per extraction experiment. This 100 mg sample of stool was extracted by using 1 ml of a novel extraction buffer.

The following extraction buffer was used:

| | | |
|---|---|---|
| 9.49 | g | Na₂HPO₄ × 2H₂O |
| 1.84 | g | KH₂PO₄ |
| 1 | g | NaN₃ |
| 0.4 | g | Na₂EDTA × 2H₂O |
| 10 | ml | chicken albumen |
| 50 | ml | Nonidet P40 10% w/v |
| 1 | tablet | Complete mini (Roche Diagnostics, Id. No. 1836145) |
| | | ad 1 l with bidistilled water |

The stool sample was extracted by shaking the tube comprising the stool specimen and the extraction buffer for approx. 15 minutes and occasionally vigorously vortexing. Thereafter the sample was centrifuged (5 min at 13.000 rpm). The supernatant of this centrifugation is called Hb extract of a stool sample or simply Hb extract.

The extract for the M2-PK measurement was prepared in the same thawed stool specimen as used for the determination of hemoglobin by using a specific sample device (Tumor M2-PK Quick-Prep, Schebo BioTech AG, Giessen) according to the package insert of the manufacturer. For this specific extraction the weighing of the stool sample was carried out by using a dosing tip, which was inserted into the feces to collect the required stool sample. The filled dosing tip was immediately transferred to the collecting tube, which contains the extraction buffer. After 10 minutes of extraction time and settling of the particles the supernatant extract, called “M2-PK extract” was ready for determination.

### EXAMPLE 3

### Immunoassays for the Determination of Hemoglobin and M2-PK from an Extract of a Stool Sample

### 3.1 Hemoglobin

The hemoglobin determination was performed with the “HaemImmun” assay (Labor Limbach, Heidelberg) according to the instructions given by the manufacturer. 10 μl, of the Hb extract was used as a sample in the immunoassay.

### 3.2 M2-PK

The determination of M2-PK was performed with the “SCHEBO Tumor M2-PK” assay (Schebo Biotech AG, Giessen) according to the instructions given by the manufacturer. 50 μL of the M2-PK-extract was used as a sample in this immunoassay.

### EXAMPLE 4

### Results

### 4.1 Sensitivity and Specificity Using the Kit Cut-Offs

From each patient two stool samples collected from different sites of a feces were measured. If one of the two stool samples revealed a positive result (if the concentration measured was found above the cut-off value), the patient sample was considered as positive.

TABLE 3
Sensitivity and specificity of Hb and M2-PK

| | Hb | M2-PK |
|---|---|---|
| Original cut-off | 2 μg/g | 4 ng/mL |
| (package insert) | | |
| Sensitivity | 58.5% | 73.4% |
| Specificity | 96.4% | 87.7% |

### 4.2 Sensitivity at a Specificity of 95% for Both Markers Individually

Due to the fact, that the M2-PK assay is too unspecific in the control group, both cut-offs were adjusted to achieve a specificity of 95%, which is considered to be a clinically relevant specificity. The results are given in table 4.

TABLE 4
Sensitivity and specificity of Hb and M2-PK with adjusted cut-offs

| | Hb | M2-PK |
|---|---|---|
| Adjusted cut-off | 0.5 μg/g | 7 ng/mL |
| Sensitivity | 72.3% | 63.8% |
| Specificity | 94.9% | 94.9% |

By adjusting the cut-offs for both assays to a specificity of about 95%, the sensitivity of Hb increased from 58.5% to 72.3 and decreased for the M2-PK assay from 73.4% to 63.8%%, respectively.

### 4.3 Sensitivity and Specificity Using Each Positive Value at the 95% Specificity Cut-Off

Additional diagnostic information can be obtained by combining the results of both assays (cf. Tables 5 and 6).

TABLE 5
Additive value by using both the measurements of Hb and
M2-PK, respectively

| CRC patients | | | Total number |
|---|---|---|---|
| (n = 94) | Hb negative | Hb positive | positive |
| M2-PK negative | 15 | 19 | |
| M2-PK positive | 11 | 49 | 60 |
| Total number | | 68 | |
| positive | | | |

The results of table 5 show that 68 samples are positive for Hb, but additional 11 samples are positive for M2-PK, which are Hb negative. If one simply would consider a single positive result either for Hb or for M2-PK to be equivalent to the presence of CRC, the total number of positive samples would be 79, which would translate to a sensitivity of 84%. This is a significantly higher sensitivity, as compared to e.g. Hb alone. However, due to the fact, that also the number of false positives is increased, the specificity is reduced to only 91.3%.

4.4 Sensitivity and Specificity after Multivariate Analysis Using RDA

To find the optimal combination of both assays, we used the regularized discriminant analysis. In this example we fixed the specificity level to 95%.

TABLE 6
Results of RDA

| | 5-fold cross-validation | | |
|---|---|---|---|
| Marker | TOTAL ERROR | Sensitivity | Specificity |
| Hb | 0.11 | 71.7% | 95.2% |
| M2-PK | 0.14 | 60.2% | 95.1% |
| Hb + M2-PK | 0.10 | 74.6% | 95.2% |

As can be seen from table 6, by combining both the measurements for Hb and M2-PK, respectively, using RDA-optimized cut-off values the aggregate specificity can be kept constant at about 95% and at the same time the diagnostic sensitivity can be increased from about 72% to about 75%.

The marker combination Hb and M2-PK in the study population investigated has a total error of only 0.10.

What is claimed is:

1. A method for assessing in vitro the absence or presence of colorectal cancer in a patient, the method comprising
providing a stool sample from the patient,
measuring in the sample a concentration of hemoglobin and a concentration of pyruvate kinase isoform M2 (M2-PK), and
correlating the concentrations measured to the concentrations of hemoglobin and M2-PK known to be associated with the absence or presence of colorectal cancer in a population.

2. The method of claim 1, further comprising the steps of measuring a concentration of a marker selected from the group consisting of carcinoembryonic antigen (CEA), a soluble fragment of cytokeratin 19 (CYFRA 21-1), carbohydrate antigen 19-9 (CA19-9), carbohydrate antigen 724 (CA72-4), nicotinamide N-methyltransferase (NNMT), pyrroline-5-carboxylate reductase (PROC), and S-adenosylhomocysteine hydrolase (SAHH) and correlating the concentration of the marker to a concentration of the marker known to be associated with the absence or presence of colorectal cancer in a population.

3. The method of claim 2 wherein the marker is SAHH.

4. A kit for performing the method of claim 1 comprising the reagents required to specifically measure hemoglobin and M2-PK in a stool sample from a patient.
